(19) United States
(12) Patent Application Publication
(10) Pub. No.: US 2015/0100062 A1
(43) Pub. Date: Apr. 9, 2015

(54) RESECTION DEVICE HAVING SUPPORT ELEMENTS AND RELATED METHODS OF USE

(71) Applicant: Boston Scientific Scimed, Inc., Maple Grove, MN (US)

(72) Inventors: Paul SMITH, Smithfield, RI (US); Samuel RAYBIN, Marlborough, MA (US); Naroun SUON, Lawrence, MA (US); Felip TORRES, Chicago, IL (US)

(21) Appl. No.: 14/507,448

(22) Filed: Oct. 6, 2014

Related U.S. Application Data
(60) Provisional application No. 61/888,216, filed on Oct. 8, 2013.

(51) Int. Cl. A61B 17/3205; A61B 17/221

(52) U.S. Cl. CPC A61B 17/32056; A61B 17/221
USPC 606/113

(57) ABSTRACT

Embodiments of the present disclosure include medical devices and related methods of use, and manufacture thereof. The medical device may include a snare including an elongate actuation member and a distal snare member. The medical device may further include a snare support including first and second arms and a base member disposed proximate to the elongate actuation member. The first arm may extend from the base member to a first lateral portion of the distal snare member and the second arm may extend from the base member to a second lateral portion of the distal snare member opposite the first lateral portion. Each arm may be moveably coupled to the distal snare member.

### CROSS REFERENCE TO RELATED APPLICATIONS

This application claims the benefit of U.S. Provisional Application No. 61/888,216, filed Oct. 8, 2013, the disclosure of which is incorporated herein by reference in its entirety.

### DESCRIPTION OF THE INVENTION

1. Technical Field

The present disclosure relates generally to medical devices and procedures. More specifically, the disclosure relates to tissue resection devices having support elements, and related methods of use and manufacture thereof.

2. Background

A wide variety of medical techniques and instruments have been developed for diagnosis as well as treatment within the body of a patient, such as in the gastrointestinal (GI) tract. Medical procedures, including Endoscopic Mucosal Resection (EMR), Endoscopic Sub-mucosal Resection (ESR), Polypectomy, Mucosectomy, etc., are minimally invasive methods for severing and retrieving malignant and non-malignant lesions, e.g., polyps. Procedures such as EMR involve resection of a lesion or unwanted tissue from a tissue wall within a body lumen. Snares have been commonly used during such medical procedures for resecting tissue from a target site. During resection procedures, physicians ensnare or capture a target tissue within a loop of the snare. Often, a physician may apply a downward force on the snare in an effort to improve snare traction around the target tissue and resect the target tissue closer to its base on the tissue wall. However, when such a downward force is applied, a distal portion of conventional snares has a tendency to deflect away from the tissue wall. Such snares may be stiff and may have insufficient traction on the tissue, and often require repeated efforts to resect the tissue before the procedure can be successfully completed. Stiffness may lead to deflection of the snare from the tissue wall, (e.g., deflection away from a tissue plane defined by the tissue wall). Also, deflection of the snare before resection can lead to an “angled cut” instead of an “even cut” (e.g., a cut substantially parallel to or coplanar with the tissue plane). As such, conventional snares conform poorly to the tissue plane during resection. Thus, there remains a need for alternative methods and systems with improved grip of the tissue to be resected.

### SUMMARY

The disclosed embodiments relate to medical devices, and related methods of use and manufacture thereof. One exemplary embodiment includes a medical device including a snare and a snare support. The snare may include an elongate actuation member and a distal snare member. The snare support may include first and second arms and a base member. The base member may be disposed proximate to the elongate actuation member. The first arm may extend from the base member to a first lateral portion of the distal snare member and the second arm may extend from the base member to a second lateral portion of the distal snare member opposite the first lateral portion. Each arm may be moveably coupled to the distal snare member.

The medical device may further include one or more of the following features: each arm may be moveably coupled to the distal snare member via a plurality of couplings; each arm may be moveably coupled to the distal snare member via a coupling configured to slide along the distal snare member; each of the couplings may be a hypotube; the base member may be fixedly coupled to the actuation member; the base member may be crimped to the actuation member; the base member may be slidably coupled to the actuation member; a plurality of stops, wherein each of the plurality of stops may be configured to limit the axial movement of the base member along the actuation member; each of the plurality of stops may be coupled with the actuation member and may extend radially outward therefrom; a sheath having a lumen, wherein each of the plurality of stops may be coupled with a wall of the sheath and may extend radially inward toward a central axis of the lumen; and each of the couplings may couple a respective arm to a medial portion of the distal snare member.

Another exemplary embodiment includes a method of operating a snare. The method may include extending a snare toward a target tissue area. The snare may include an elongate actuation member and a distal snare member. The method may further include positioning the distal snare member about the target tissue area. Also, the method may include supporting the distal snare member via a snare support. The snare support may include first and second arms and a base member disposed proximate to the elongate actuation member. The first arm may extend from the base member to a first lateral portion of the distal snare member and the second arm may extend from the base member to a second lateral portion of the distal snare member opposite the first lateral portion. Each arm may be moveably coupled to the distal snare member. Further, the method may include cutting tissue of the target tissue area.

The method may further include one or more of the following features: each arm may be moveably coupled to the distal snare member via a coupling and the method may further include sliding each of the couplings along the distal snare member; each of the couplings may be a hypotube; the base member may be fixedly coupled to the actuation member; the base member may be slidably coupled to the actuation member; and moving the base member longitudinally along the actuation member between a plurality of axial stops so as to at least one of urge and retract the arms along the distal snare member.

Another exemplary embodiment includes a medical device. The medical device may include a snare and a snare support. The snare may include an elongate actuation member and a distal operating member. The snare support may be configured to vary the stiffness of the operating member. The snare support may include a first arm, a second arm, and a base member. Each of the first and second arms may be fixedly coupled to the base member and moveably coupled to the distal operating member via a coupler. The base member may be moveably coupled to the elongate actuation member.

The medical device may further include one or more of the following features: a plurality of axial stops may be configured to limit axial movement of the base member relative to the actuation member, and each of the plurality of stops may be coupled with the actuation member and may extend radially outward therefrom; and a plurality of axial stops may be configured to limit axial movement of the base member relative to the actuation member, and a sheath having a lumen, wherein each of the plurality of stops may be coupled with a wall of the sheath and may extend radially inward toward a central axis of the lumen.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 schematically illustrates an exemplary medical device, according to an embodiment of the present disclosure;

FIG. 2 is a partial view of the exemplary medical device of FIG. 1, illustrating a snare support element;

FIG. 3 is another partial view of the exemplary medical device of FIG. 1, illustrating a base member, according to an embodiment of the present disclosure;

FIG. 4 is a partial view of the exemplary medical device of FIG. 1, employing one or more stops;

FIG. 5 shows one exemplary stop, according to an embodiment of the present disclosure; and

FIG. 6 illustrates an exemplary sheath employing one or more stops.

### DETAILED DESCRIPTION OF EMBODIMENTS

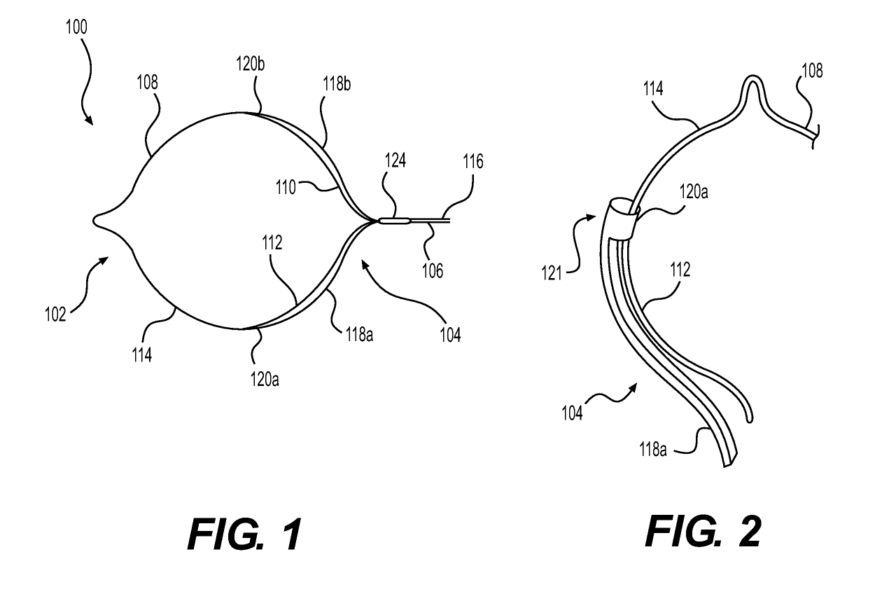

FIG. 1 shows an exemplary medical device 100 such as a snare support system that may include a snare 102 and a snare support 104. As shown, the snare 102 may include a push/pull wire 106 and a distal member/snare member (e.g., operating member) 108. The snare member 108 may have a proximal portion 110, distal portion 114, and medial/lateral portion 112 extending between the proximal portion 110 and distal portion 114. In general, the snare member 108 may be delivered to a target tissue via an introduction sheath (e.g., sheath 300 shown in FIG. 6). In some embodiments, the sheath may be a catheter, endoscope, a sheath independent of any other device, and/or any other appropriate hollow structure configured to be inserted and manipulated within the patient's body as known in the art. The sheath may have a substantially circular cross-sectional shape, but other suitable cross-sectional shapes such as elliptical, oval, polygonal, or irregular may also be employed.

The snare member 108 may be configured to transition between a first collapsed configuration within a lumen of the sheath, and a second expanded configuration in which the snare member 108 may be caused to extend outwards from a distal opening of the sheath. That is, in the collapsed configuration, the snare member 108 may be compressed within the lumen of the sheath. Upon proper positioning within a patient's body such that a distal end of the sheath is disposed adjacent a target tissue area, a physician may extend the snare member distally of (e.g., outward and away from) the sheath. Upon such action, the snare member 108 may be relieved of the compression forces provided by the sheath, As such, the snare member 108 may be free to transition into the expanded configuration. During use, the snare member 108 may be caused to encircle the target tissue when in the expanded configuration. Thereafter, the snare member 108 may be tightened about target tissue by retracting the snare member 108 into the sheath or the like such that the snare member 108 may transition from the expanded configuration towards the collapsed configuration.

A push/pull wire 106 may be operable to extend and retract the snare member 108 such that the snare member 108 may transition between the expanded and the collapsed configurations. The push/pull wire 106 may have a proximal end (not shown), and a distal end 116. The proximal end of the push/pull wire 106 may be connected to a handle (not shown), while the distal end 116 may be coupled to the proximal portion 110 of the snare member 108. Specifically, the push/pull wire 106 (sometimes also referred to as legs of the snare member 108) may connect the snare member 108 to a handle (not shown) located on a proximal end (not shown) of the medical device 100 outside the patient's body. The push/pull wire 106 may extend and retract the snare member 108 and thereby transition the snare member 108 between the collapsed configuration and expanded configuration. For example, when a user applies an axial force in a distal direction via the handle, the push/pull wire 106 may extend (e.g., push) the snare member 108 outwards from the sheath (e.g., sheath 300 shown in FIG. 6), thus moving the snare member 108 into the expanded configuration. Similarly, when a user applies an axial force in a proximal direction via the handle, the push/pull wire 106 may retract (e.g., pull) the snare member 108 into the sheath, thus moving the snare member 108 into the collapsed configuration. In some embodiments, the push/pull wire 106 and the snare member 108 may be made of a continuous, e.g., monolithically formed unitary structure. However, in other embodiments, the push/pull wire 106 and the snare member 108 may be discrete and coupled to each other using any suitable technique known in art. Exemplary techniques may include welding, soldering, and/or heat bonding. While described as a wire, the push/pull wire 106 may be comprised of a wire, or any other suitable elongate structure (e.g., elongate actuation member).

The snare member 108 may be a wire in the form of a loop configured for surrounding and thereby severing and/or retrieving undesirable tissue. In some embodiments, the snare 102, including the snare member 108, can be a monofilament wire or a multifilament wire. The multifilament wire may be bonded, twisted together, crimped, and/or braided. In the illustrated embodiment, the snare member 108 may form a substantially circular loop shape. However, in some embodiments, the snare member 108 may be configured to have any suitable loop shape such as, but not limited to, oval, hexagonal, rectangle, square, irregular, polygonal, semicircular, octagonal, or the like. Further, the snare member 108 can be formed using any suitable biocompatible material such as, but not limited to metals, polymers, alloys, or the like. Exemplary materials may include steel, tungsten, NITINOL, or titanium, and so forth.

In some embodiments, the snare member 108 may be configured for electro-cauterization procedures. In such embodiments, the snare member 108 and the push/pull wire 106 may be formed from suitable conducting material, e.g., stainless steel or NITINOL. The snare member 108 may be configured to transmit electric current within specific ranges and be able to withstand repeated heating cycles during electro-cauterization procedure.

As shown in FIG. 1, the snare member 108 may be supported via the snare support 104 to increase the overall stiffness of the snare member 108, without increasing the stiffness at the distal portion 114 of the snare member 108. For example, the snare support 104 may be coupled to the snare member 108 so as to increase the stiffness of the snare member 108 at the proximal portion 110 and/or medial/lateral portion 112, and may simultaneously provide adequate flexibility at the distal portion 114 to enable improved capture and resection of the target tissue. The snare support 104 may be moveably (e.g., slidably) connected to the proximal portion 110 or medial portion 112 of the snare member 108. More particularly, the snare support 104 may include two arms 118a, 118b, (collectively 118) coupled to the snare member 108 at diametrically opposite locations. In one embodiment, as shown in FIG. 1, the arms 118 may extend substantially alongside the snare member 108 when the snare member 108 is in the fully expanded configuration such that the arms 118 and snare member 108 have substantially the same curvature. As used in this application, the term “substantially” means within plus or minus 5%. The arms 118 may be coupled to the snare member 108 such that the arms 118 may move (e.g., slide) relative to the snare member 108. For example, as shown in FIG. 1, each of arms 118 are configured to axially slide or translate along the snare member 108 in the proximal and distal directions. To facilitate this, each of the arms 118 may include a moveable coupling/coupler 120a, 120b (collectively 120). Additionally, the snare support 104 may include a base member 124 configured for receiving the arms 118. In some embodiments, during axial retraction of the snare member 108 and snare support 104 into the sheath, as will be described in greater detail below, the arms 118 may move toward or away from the snare member 108 such that the arms 118 no longer extend substantially alongside with the snare member 108. In such a manner, snare support 104 may assist in maintaining the desired shape of the snare member 108 during retraction into the sheath.

FIG. 2 illustrates a partial view of the snare support 104 coupled to the snare member 108 as shown in FIG. 1. As noted above, the snare support 104 may allow for varied stiffness between the proximal 110 and distal portions 114 of the snare member 108. In general, the snare support 104 may include two arms 118 coupled to the lateral or medial portion 112 of the snare member 108. More particularly, FIG. 2 illustrates arm 118a coupled to one side of the snare member 108. While FIG. 2 depicts one side of the snare member 108, and therefore only one arm 118a, it is understood that a second arm 118b is coupled to a second side of snare member 108 as shown in FIG. 1.

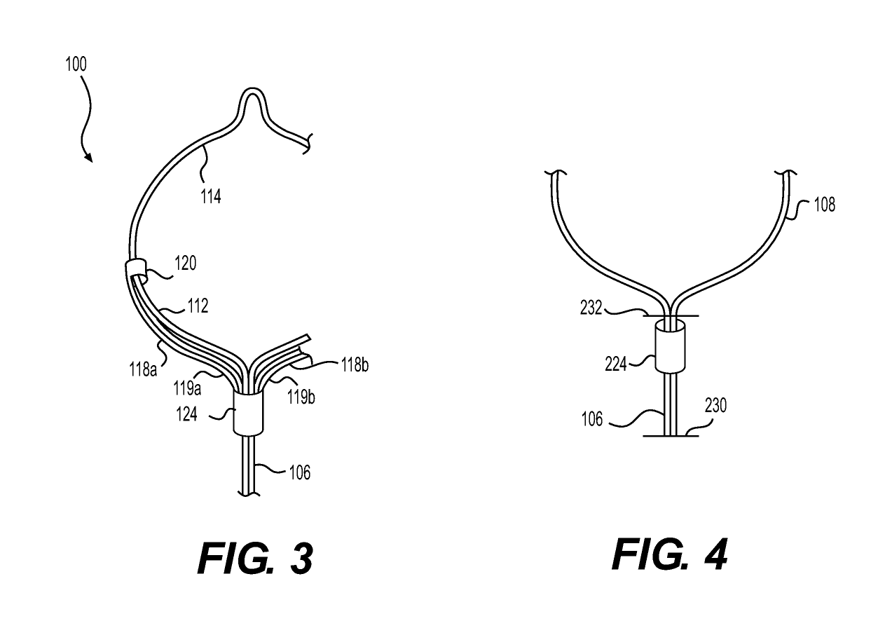

Each arm 118 may have a proximal end 119 (shown in FIG. 3), a distal end 121, and may extend outward along, and in some embodiments, substantially parallel to the snare member 108 when the snare member 108 is in the fully expanded configuration. The distal end 121 of each arm 118 may be coupled to the medial portion 112 of the snare member 108 via coupler 120, while the proximal end 119 may be coupled to the base member 124 (see FIGS. 1 and 3). In some embodiments, each arm 118 may be coupled to the snare member 108 along the medial portion 112. Alternatively, each arm 118 may be coupled to the snare member 108 along the proximal portion 110. In other words, each arm 118 may be coupled to the snare member 108 at any position proximate the distal portion 114 of the snare member 108 so as to allow for increased flexibility of the distal portion 114.

As noted above, each arm 118a, 118b may be coupled to the snare member 108 such that the arms 118 may move, e.g., axially slide or translate relative to the snare member 108. As shown in FIG. 2, for example, arm 118a may be configured to slide relative to snare member 108 via coupling 120a disposed on the distal end 121 of the arm 118a. As such, arms 118 may dynamically vary the stiffness of the snare member 108 during use. For example, as snare member 108 is caused to move towards the collapsed configuration (e.g., when snare member is axially retracted in the proximal direction via the push/pull wire 106), snare member 108 and arms 118 may be caused to move relative to one another. As arms 118 move along the snare member 108, the arms 118 may increase stiffness of the snare member 108 at coupler 120. In this manner, the increased stiffness of the snare member 108 may result in improved control for the physician during a procedure. Additionally, as the distal portion 114 of snare member 108 is free from interaction with snare support 104 including arms 118, the distal portion 114 is provided sufficient relative flexibility to better conform to the tissue wall during a resection procedure. In contrast to conventional snares, the disclosed embodiments provide a snare member 108 in which a distal portion 114 of the snare member 108 is sufficiently flexible so as to conform to the tissue wall while proximal 110 and/or medial 112 portions of the snare member 108 can be made sufficiently stiff to enable better control by a physician. For example, in the fully expanded configuration, arms 118 may provide increased relative stiffness to snare member 108 at least along the portion of snare member 108 at which the arms 118 are coupled to the snare member 108. As the snare member 108 is moved between the fully expanded and fully collapsed configurations, the location of the coupler 120 may move relative to the snare member 108 thereby dynamically changing the relative stiffness of snare member 108 at least along the portion of snare member 108 at which the arms 118 are coupled to the snare member 108.

By way of example only, in one embodiment, as shown in FIG. 1, the arms 118 may be coupled to the snare member 108 at the medial portion 112. Accordingly, in this arrangement, approximately half of the snare member 108 (e.g., the portion of the snare member 108 proximal of couplers 120a and 120b) may exhibit increased stiffness relative to the remainder half of the snare member 108 (e.g., the portion of the snare member 108 distal of couplers 120a and 120b).

In the illustrated embodiments, a single coupling 120 is shown to couple each arm 118 to the snare member 108. However, in alternative embodiments, two or more couplings 120 may be utilized to couple each arm 118 to the snare member 108 at multiple points to further enhance the stiffness of the snare member 108. In other words, each arm 118 may have multiple couplers 120 coupling the arm 118 to the snare member 108 at multiple points.

Each coupler 120 may include any appropriate connection configured to move relative to snare member 108. For example, each coupler 120 may comprise a hypotube or any other appropriate hollow structure surrounding the snare member 108 and connected to an arm 118. Other structural variations may also be contemplated. In some embodiments, the coupler 120 may be a separate, discrete component coupled to the arm 118 via any suitable techniques such as soldering, welding, adhesive bonding, crimping or the like, while in other embodiments, the coupling 120 may be integrally (e.g., monolithically formed from a single continuous piece of material) with the arm 118.

The arms 118 may generally be an elongated and rigid structure. In some embodiments, the arms 118 may be formed of flat wire or sheet material with a rectangular cross-section providing suitable stiffness to enhance controllability and traction of the snare member 108. For example, the arms 118 may be stamped so as to allow bending of the arms 118 towards/away from a center of the snare member 108 while preventing twisting motion during use. In this way, undesired deflection of the snare member 108 relative to the tissue plane may be avoided. For example, the arms 118 may assist the operator/physician in proper placement of the snare member 108 about the target tissue by supporting the snare member 108 during placement, and preventing unintended deflection (or twisting) away from the tissue plane during a procedure. Other cross-sectional shapes such as circular, oval, polygonal, or the like may also be contemplated. Additionally, the cross-sectional shape of the arms 118 may vary based on the cross-sectional shape of the snare member 108. In general, any suitable biocompatible materials known in the art, such as Nitinol, stainless steel, or polyimide may be employed to manufacture the arms 118. The chosen material may be based on desired stiffness, resilience, and other properties, as will be understood by those skilled in the art. In some embodiments, the arm 118 may be conductive or nonconductive, and may be made of any suitable materials such as steel alloy, NITINOL, polymer, cobalt chromium, tungsten, or other material.

FIG. 3 shows a partial view of the medical device 100, illustrating a base member 124. The base member 124 may be a hypotube, collar, or any appropriate hollow structure configured to receive proximal ends 119a, 119b of arms 118a, and 118b, respectively. The arms 118 may be coupled to the base member 124 via welding, adhesives, stamping, crimping or the like. Alternatively, the base member 124 and arms 118 may be monolithically formed from a single continuous piece of material. The base member 124 may be fixedly or slidably (e.g., see FIG. 4) coupled to the push/pull wire 106.

In the illustrated embodiment of FIG. 3, the base member 124 may be fixedly coupled to the push/pull wire 106. To this end, the base member 124 may be crimped with the push/pull wire 106 and arms 118. Such a rigid (e.g., non-movable) connection may provide stiffness at the proximal portion 110 of the snare member 108. In some embodiments, the base member 124 may be positioned adjacent to the proximal portion 110 of the snare member 108. Alternatively, the base member 124 may be positioned at a pre-defined distance proximate of the proximal portion 110 of the snare member 108.

In some embodiments, as shown in FIG. 4, a base member 224 may be moveable (e.g., axially slidable) relative to the push/pull wire 106. In such an embodiment, the base member 224 may slide along a portion of the push/pull wire 106. In this arrangement, the push/pull wire 106 may include one or more stops 230, 232 preventing axial displacement of base member 224, and thereby, snare support 104 including arms 118. For example, a proximal stop 230 may be employed to limit the proximal movement of the base member 224, while a distal stop 232 may be employed to limit the distal movement of the base member 224. As such, the base member 224 may travel longitudinally along the push/pull wire 106 between the proximal stop 230 and the distal stop 232, to urge and retract the arms 118 along the snare member 108. The stops 230, 232, may extend radially outward from the push/pull wire 106. While FIG. 4 illustrates stops 230 and 232 schematically, it is understood that any appropriate geometric configuration sufficient to arrest axial movement of the base member 224 may be used. Additionally, the stops 230, 232, may be coupled to the push/pull wire 106 via any appropriate manner, including, for example, adhesives, heat bonding, welding, soldering, screw connections, or the like.

The stops 230, 232 may represent any elongated structure capable of restricting or limiting the movement of the base member 224 along the push/pull wire 106. In some cases, the stops 230, 232 can have circular cross-sections, while other cross-sectional shapes such as oval, rectangular, semi-circular, or the like may also be contemplated. In some embodiments, the stops 230, 232 may include any suitable structure such as protrusions to control the movement of the base member 224 along the length of the push/pull wire 106.

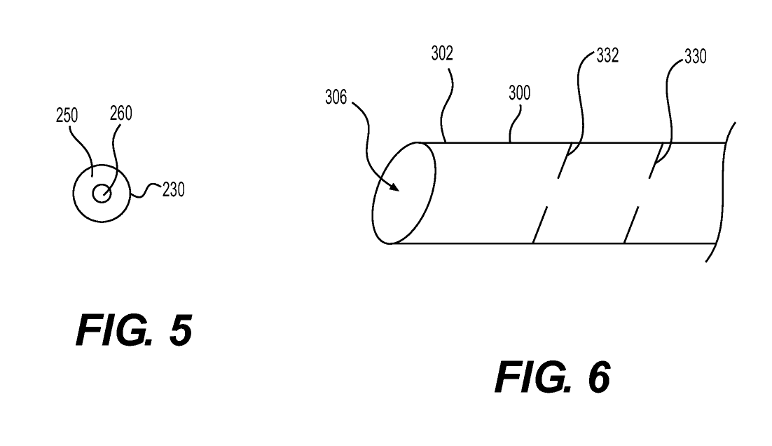

FIG. 5 illustrates an exemplary stop 230, according to an embodiment of the present disclosure. The stop 230 may include solid member 250 and a lumen 260 through which the push/pull wire 106 may pass and extend to the proximal end of the medical device 100. As discussed above, the stop 230 may be configured to limit the axial movement of the base member 224 coupled to the push/pull wire 106, thereby limiting the movement of the arms 118 along the snare member 108. In the illustrated embodiment, the stop 230 may be a circular disc shape member having a concentric lumen. Alternatively, the stop 230 may include any alternative shape having any lumen therethrough configured to receive the push/pull wire 106. For example, in one alternative embodiment, the solid member 250 may be a circular disc shape member but with a different shaped lumen 260 (e.g., rectangular, oblong, elongated, or the like) extending therethrough. Stops 230, 232 may include metals, polymers, and alloys such as NITINOL, polycarbonate, stainless steel, PVC, or the like.

In alternative embodiments, it may be desirable to limit axial motion of base member 224 without the inclusion of stops 232 and 230 on the push/pull wire 106. In such embodiments, similar stops may be included within the sheath, for example, sheath 300 shown in FIG. 6. FIG. 6 illustrates an exemplary sheath 300 employing one or more stops s 330, 332 such as a proximal stop 330 and a distal stop 332. The sheath 300 may have a proximal end (not shown), distal end 302, and a lumen 306 extending therebetween. The proximal and distal stops 330, 332 may be coupled with a wall of the sheath 300 and may extend radially inward toward the central axis of the lumen 306. The proximal and distal stops 330, 332, may be coupled to the sheath 300 such that the axial movement of the base member 224 may be limited in between the proximal and distal stops 330, 332. In some embodiments, the stops 330, 332 may be integrally formed with the sheath 300 via known techniques such as molding, etc.

During operation, the snare support system 100 including the snare 102 and snare support 104 may be inserted into the sheath in the fully collapsed configuration. Upon assembly, the sheath (e.g., sheath 300 of FIG. 6) may be introduced into the patient and positioned such that a distal end of the sheath is proximate a target tissue. Once in position, the physician may actuate the push/pull wire 106 such that the snare member 108 may be extended out of the distal end of the sheath. As such, the snare member 108 may transition from the fully collapsed configuration to the expanded configuration. Once deployed, the physician may manipulate the snare member 108 so as to encircle the target tissue. In the fully expanded configuration, the arms 118 may extend parallel to the snare member 108. After positioning the snare member 108 about (e.g., around) the target tissue, the physician may transition the snare member 108 from the expanded configuration toward the collapsed configuration to capture and resect the target tissue. As the snare member 108 moves from the fully expanded configuration towards the collapsed configuration, the arms 118 may move relative to the snare member 108 so as to dynamically vary the stiffness of the snare member 108, as discussed above. After the tissue is resected, the snare support system 100 (including snare member 108, and snare support 104) may be retracted back into the sheath. Finally, the sheath may be retracted from the patient's body.

The disclosed embodiments enable a physician to maintain better control of a snare during a resection procedure. For example, in contrast to conventional snares, the disclosed embodiments including the snare support 104 are particularly advantageous for maintaining a desired shape of the snare member 108. For example, the snare member 108 may maintain the desired shape, e.g., circular shape as shown in FIGS. 1-4, longer while be retracted into a sheath. In other words, since the arms 118 provide added support to the snare member 108, the snare member 108 is enabled to maintain its desired shape for a longer range of motion between the fully expanded configuration and the collapsed configuration within the sheath. In some embodiments, in the fully expanded configuration, the arms may extend substantially parallel with the snare member 108. Upon axial retraction into the sheath, the snare member 108 may resist collapsing for a longer period of time while being drawn into the sheath. In such an arrangement, during retraction of the snare member 108 into the sheath, the arms 118 may bend toward or away from the snare member 108 such that they no longer extend substantially parallel with the snare member 108. As such, the arms 118 may bias and/or leverage the snare member 108 towards its desired shape thereby resisting collapse.

Additionally, as noted above, the disclosed embodiments provide improved stiffness variation along the snare member 108 which may result in improved control for the physician during a procedure. As the distal portion 114 of snare member 108 is free from interaction with snare support 104 including arms 118, the distal portion 114 is provided sufficient relative flexibility to better conform to the tissue wall during a resection procedure. In other words, in contrast to conventional snares, the disclosed embodiments provide a snare member 108 in which a distal portion 114 of the snare member 108 is sufficiently flexible so as to conform to the tissue wall while proximal 110 and/or medial 112 portions of the snare member 108 are sufficiently stiff to enable better control by a physician.

Although the embodiments described above have been disclosed in connection with devices for manipulating tissues, those skilled in the art will understand that the principles set out above can be applied to any tissue resection device and can be implemented in different ways without departing from the scope of the disclosure as defined by the claims. In particular, constructional details, including manufacturing techniques and materials, are well within the understanding of those of skill in the art and have not been set out in any detail here. These and other modifications and variations are well within the scope of the present disclosure and can be envisioned and implemented by those of skill in the art.

Other embodiments of the present disclosure will be apparent to those skilled in the art from consideration of the specification and practice of the embodiments disclosed herein. It is intended that the specification and examples be considered as exemplary only, and departures in form and detail may be made without departing from the scope and spirit of the present disclosure as defined by the following claims.

What is claimed is:

1. A medical device, comprising:
a snare including an elongate actuation member and a distal snare member; and
a snare support including first and second arms and a base member disposed proximate to the elongate actuation member, the first arm extending from the base member to a first lateral portion of the distal snare member, and the second arm extending from the base member to a second lateral portion of the distal snare member opposite the first lateral portion, wherein each arm is moveably coupled to the distal snare member.

2. The device of claim 1, wherein each arm is moveably coupled to the distal snare member via a plurality of couplings.

3. The device of claim 1, wherein each arm is moveably coupled to the distal snare member via a coupling configured to slide along the distal snare member.

4. The device of claim 3, wherein each of the couplings is a hypotube.

5. The device of claim 1, wherein the base member is fixedly coupled to the actuation member.

6. The device of claim 5, wherein the base member is crimped to the actuation member.

7. The device of claim 1, wherein the base member is slidably coupled to the actuation member.

8. The device of claim 7, further including a plurality of stops, wherein each of the plurality of stops is configured to limit the axial movement of the base member along the actuation member.

9. The device of claim 8, wherein each of the plurality of stops is coupled with the actuation member and extends radially outward therefrom.

10. The device of claim 8, further including:
a sheath having a lumen, wherein each of the plurality of stops is coupled with a wall of the sheath and extends radially inward toward a central axis of the lumen.

11. The device of claim 3, wherein each of the couplings couples a respective arm to a medial portion of the distal snare member.

12. A method of operating a snare, comprising:
extending a snare toward a target tissue area, the snare including an elongate actuation member and a distal snare member;
positioning the distal snare member about the target tissue area;
supporting the distal snare member via a snare support including first and second arms and a base member disposed proximate to the elongate actuation member, the first arm extending from the base member to a first lateral portion of the distal snare member, and the second arm extending from the base member to a second lateral portion of the distal snare member opposite the first lateral portion, wherein each arm is moveably coupled to the distal snare member; and
cutting tissue of the target tissue area.

13. The method of claim 12, wherein each arm is moveably coupled to the distal snare member via a coupling, the method further including:
sliding each of the couplings along the distal snare member.

14. The method of claim 13, wherein each of the couplings is a hypotube.

15. The method of claim 12, wherein the base member is fixedly coupled to the actuation member.

16. The method of claim 12, wherein the base member is slidably coupled to the actuation member.

17. The method of claim 16, further including:
moving the base member longitudinally along the actuation member between a plurality of axial stops so as to at least one of urge and retract the arms along the distal snare member.

18. A medical device, comprising:
a snare including an elongate actuation member and a distal operating member; and
a snare support configured to vary the stiffness of the operating member, the support including a first arm, a second arm, and a base member, each of the first and second arms fixedly coupled to the base member and moveably coupled to the distal operating member via a coupler, wherein the base member is moveably coupled to the elongate actuation member.

19. The device of claim 18, further comprising:
a plurality of axial stops configured to limit axial movement of the base member relative to the actuation member, wherein each of the plurality of stops is coupled with the actuation member and extends radially outward therefrom.

20. The device of claim 18, further including:
a plurality of axial stops configured to limit axial movement of the base member relative to the actuation member; and
a sheath having a lumen, wherein each of the plurality of stops is coupled with a wall of the sheath and extends radially inward toward a central axis of the lumen.
